(19) United States
(12) Patent Application Publication
(10) Pub. No.: US 2015/0202218 A1
(43) Pub. Date: Jul. 23, 2015

(54) Broad Spectrum Inhibitors of the Post Proline Cleaving Enzymes for Treatment of Hepatitis C Virus Infections

(71) Applicant: Trustees of Tufts College, Boston, MA (US)

(72) Inventor: William W. Bachovchin, Cambridge, MA (US)

(21) Appl. No.: 14/416,220

(22) PCT Filed: Aug. 1, 2013
(86) PCT No.: PCT/US2013/053167

§ 371 Date: Jan. 21, 2015

Related U.S. Application Data
(60) Provisional application No. 61/678,798, filed on Aug. 2, 2012.

(51) Int. Cl. A61K 31/69; A61K 45/06

(52) U.S. Cl. CPC A61K 31/69; A61K 45/06

(57) ABSTRACT

Disclosed are methods of treating, inhibiting, or preventing a viral infection in a mammal in need thereof by administering a therapeutically or prophylactically effective amount of an inhibitor of FAP, an inhibitor of DPPIV, an inhibitor of DPP8, or an inhibitor of DPP9. The inhibitor may act as both an inhibitor of DPPIV and an inhibitor of DPP8/9. The viral infection includes, but is not limited to, hepatitis B virus, hepatitis C virus, human immunodeficiency virus, Polio virus, Coxsackie A virus, Coxsackie B virus, Rhino virus, respiratory syncytial virus, dengue virus, equine infectious anemia virus, Echo virus, small pox virus, Ebola virus, and West Nile virus.

### RELATED APPLICATIONS

This application claims the benefit of priority to U.S. Provisional Patent Application Ser. No. 61/678,798, filed Aug. 2, 2012.

### BACKGROUND OF THE INVENTION

Hepatitis C virus (HCV) is a major health problem; there are approximately 170 million infected individuals worldwide. HCV primarily affects the liver. Chronic infection can lead to scarring of the liver and ultimately to cirrhosis, which is generally only apparent after many years. In some cases, individuals with cirrhosis will go on to develop liver failure, liver cancer, or life-threatening esophageal or gastric varices.

Unfortunately, a reliable cure for chronic HCV infection has remained elusive. However, if a patient receives pegylated interferon (IFN) and ribavirin, he or she can sometimes clear the virus via sustained anti-viral immune response. In other words, often the virus eludes the available therapeutic strategies.

CXCL10, also known as interferon inducible protein-10 or IP-10, mediates chemoattraction of activated lymphocytes to the liver and was, therefore, viewed as a positive prognostic biomarker for anti-viral therapy and anti-viral immune responses. However, CXCL10 has recently been validated as a negative prognostic biomarker in HCV infection. Casrouge, A. et al. J. Clin. Invest. 2011; 121(1): 308-317. This counterintuitive result has been explained in terms of a truncated form of IP-10 produced by the action of a post proline cleaving enzyme known as dipeptidyl peptidase type 4 (DPPIV). HCV often causes liver cirrhosis, and liver cirrhosis is known to correlate with upregulation of DPPIV. The additional DPPIV, in turn, acts to cleave the N-terminal Val-Pro dipeptide from CXCL10, which transforms CXCL10 into an antagonist of lymphocyte chemoattraction to the liver, contributing to the ability of HCV to elude the immune response.

There exists a need for small molecule drugs with potent activity against HCV.

### SUMMARY OF THE INVENTION

In certain embodiments, the invention relates to a method of increasing CXCL10 secretion by a cell, comprising the step of contacting the cell with an inhibitor of FAP, an inhibitor of DPPIV, an inhibitor of DPP8, or an inhibitor of DPP9.

In certain embodiments, the invention relates to a method of treating, inhibiting, or preventing a viral infection, comprising the step of administering to a mammal in need thereof a therapeutically or prophylactically effective amount of an inhibitor of FAP, an inhibitor of DPPIV, an inhibitor of DPP8, or an inhibitor of DPP9.

In certain embodiments, the invention relates to any one of the aforementioned methods, wherein the inhibitor is an inhibitor of FAP and an inhibitor of DPPIV.

In certain embodiments, the invention relates to any one of the aforementioned methods, wherein the inhibitor is an inhibitor of FAP and an inhibitor of DPP8.

In certain embodiments, the invention relates to any one of the aforementioned methods, wherein the inhibitor is an inhibitor of FAP and an inhibitor of DPP9.

In certain embodiments, the invention relates to any one of the aforementioned methods, wherein the inhibitor is an inhibitor of DPP8 and an inhibitor of DPPIV.

In certain embodiments, the invention relates to any one of the aforementioned methods, wherein the inhibitor is an inhibitor of DPP9 and an inhibitor of DPPIV.

In certain embodiments, the invention relates to any one of the aforementioned methods, wherein the inhibitor is an inhibitor of DPP8 and an inhibitor of DPP9.

In certain embodiments, the invention relates to any one of the aforementioned methods, wherein the inhibitor is an inhibitor of FAP, an inhibitor of DPPIV, and an inhibitor of DPP8.

In certain embodiments, the invention relates to any one of the aforementioned methods, wherein the inhibitor is an inhibitor of FAP, an inhibitor of DPPIV, and an inhibitor of DPP9.

In certain embodiments, the invention relates to any one of the aforementioned methods, wherein the inhibitor is an inhibitor of FAP, an inhibitor of DPP8, and an inhibitor of DPP9.

In certain embodiments, the invention relates to any one of the aforementioned methods, wherein the inhibitor is an inhibitor of DPPIV, an inhibitor of DPP8, and an inhibitor of DPP9.

In certain embodiments, the invention relates to any one of the aforementioned methods, wherein the viral infection is a viral infection of the liver.

In certain embodiments, the invention relates to any one of the aforementioned methods, wherein the viral infection is hepatitis B virus, hepatitis C virus, human immunodeficiency virus, Polio virus, Coxsackie A virus, Coxsackie B virus, Rhino virus, respiratory syncytial virus, dengue virus, equine infectious anemia virus, Echo virus, small pox virus, Ebola virus, or West Nile virus.

In certain embodiments, the invention relates to any one of the aforementioned methods, wherein the viral infection is hepatitis C virus.

In certain embodiments, the invention relates to any one of the aforementioned methods, wherein the inhibitor has a structure of Formula (I)

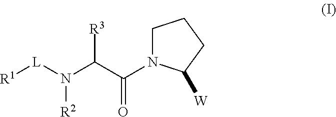

wherein
L is absent or is —XC(O)—;
R¹ is selected from H, C₁₋₆alkyl, C₁₋₆acyl, C₁₋₆aralkyl, C₁₋₆aracyl, C₁₋₆heteroaracyl, carbocyclyl, aryl, and ArSO₂—;
R² is selected from H and C₁₋₆alkyl, or R¹ and R² together are phthaloyl, thereby forming a ring;
R³ is selected from H, C₁₋₆alkyl, C₁₋₆hydroxyalkyl, C₁₋₆thioalkyl, and C₁₋₆aralkyl;
W is selected from B(Y¹)(Y²) and CN;
Y¹ and Y² are independently selected from OH or a group that is hydrolyzable to give a boronic acid, or together with the boron atom to which they are attached form a 5- to 8-membered ring that is hydrolyzable to a boronic acid;
X is selected from O and NH.

In certain embodiments, the invention relates to any one of the aforementioned methods, wherein the inhibitor has a structure of Formula (II)

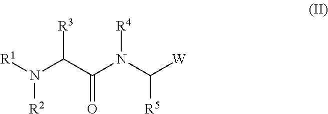

wherein
R¹ is selected from H, C₁₋₆alkyl, C₁₋₆acyl, C₁₋₆aralkyl, C₁₋₆aracyl, C₁₋₆heteroaracyl, and carbocyclyl;
R² is selected from H and C₁₋₆alkyl;
R³ is selected from H, C₁₋₆alkyl, C₁₋₆hydroxyalkyl, C₁₋₆thioalkyl, and C₁₋₆aralkyl;
R⁴ is selected from H and C₁₋₆alkyl, or R³ and R⁴ together are C₁₋₆alkyl thereby forming a ring;
R⁵ is selected from H, C₁₋₆alkyl, C₁₋₆hydroxyalkyl, C₁₋₆thioalkyl, and C₁₋₆aralkyl, or R⁴ and R⁵ together are C₁₋₆alkyl-S—C₁₋₆alkyl;
W is selected from H, B(Y¹)(Y²), and CN;
Y¹ and Y² are independently selected from OH or a group that is hydrolyzable to give a boronic acid, or together with the boron atom to which they are attached form a 5- to 8-membered ring that is hydrolyzable to a boronic acid;
   with the proviso that W can be H only when R⁴ and R⁵ together are C₁₋₆alkyl-S—C₁₋₆alkyl.

In certain embodiments, the invention relates to any one of the aforementioned methods, wherein the inhibitor is selected from the group consisting of:

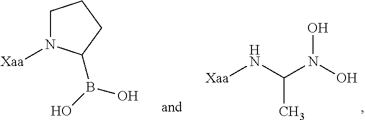

wherein Xaa is a natural or non-natural amino acid.

In certain embodiments, the invention relates to any one of the aforementioned methods, wherein the inhibitor is selected from the group consisting of:

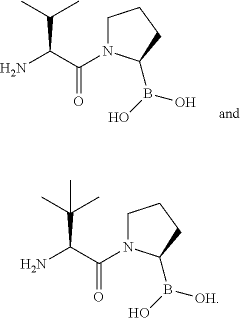 [SMILES: CC(C)[C@H](N)C(=O)N1CCC[C@H]1B(O)O ; CC(C)(C)[C@H](N)C(=O)N1CCC[C@H]1B(O)O]

In certain embodiments, the invention relates to any one of the aforementioned methods, wherein the inhibitor is described in U.S. Patent Application Publication No. 2010/0184706, U.S. Patent Application Publication No. 2010/0105753, U.S. Patent Application Publication No. 2010/0105629, U.S. Patent Application Publication No. 2009/0209491, U.S. Patent Application Publication No. 2009/0124559, U.S. Patent Application Publication No. 2005/0203027, U.S. Pat. No. 7,998,997, U.S. Pat. No. 7,727,964, U.S. Pat. No. 7,691,967, U.S. Pat. No. 7,459,428, and U.S. Pat. No. 6,011,155, the contents of each of which are incorporated by reference in their entirety.

In certain embodiments, the invention relates to any one of the aforementioned methods, wherein the inhibitor is sitagliptin, vildagliptin, saxagliptin, linagliptin, dutoglipin, gemigliptin, alogliptin, or berberine.

### BRIEF DESCRIPTION OF THE FIGURE

FIG. 1 depicts a new CXCL10 paradigm in hepatitis C. CXCL10 and DPP4 levels rise following HCV infection. DPP4 converts the active form of CXCL10 into a shorter form that is an antagonist rather than an agonist of the cognate receptor CXCR3. The short CXCL10 predominates and probably impedes CXCR3-mediated lymphocyte recruitment to the infected liver. This rationale logically explains the association between increased CXCL10 levels and poor antiviral response to TN-based therapy. The cell types that make CXCL10 or DPP4 are depicted. DPP4 is shed from cell surfaces, Cells and structures are not shown to scale.

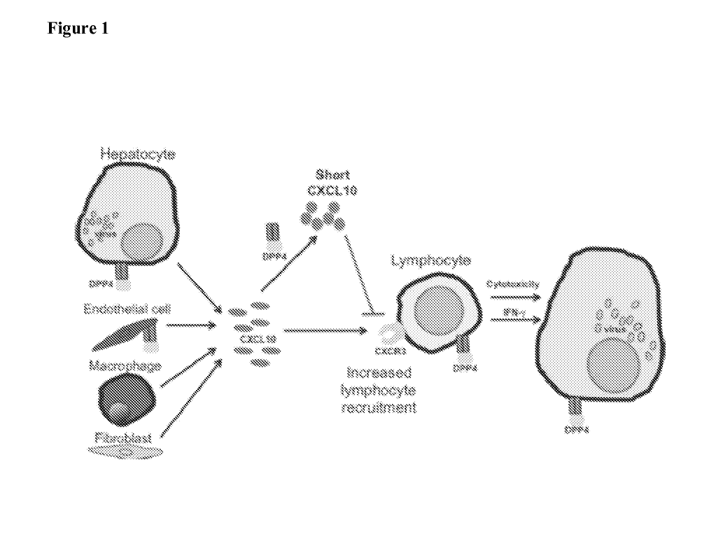

### DETAILED DESCRIPTION OF THE INVENTION

### Overview

In certain embodiments, the invention relates to methods of treating viral infections, including HCV infections, by simultaneously stimulating production of CXCL10 and preventing the conversion of CXCL10 to an antagonist. In certain embodiments, the invention relates to any one of the aforementioned methods, comprising administering one or more compounds that inhibit DPP8/9 or DPPIV or FAP, or any combination thereof.

### Chemokines

The chemokines CXCL9, CXCL10, and CXCL11—also known as monokine induced by interferon-γ, interferon-inducible protein-10, and interferon-inducible T-cell α-chemoattractant, respectively—are structurally and functionally related molecules. These chemokines are generally not detectable in most non-lymphoid tissues under physiological conditions, but are strongly induced by cytokines, particularly interferon-γ, during infection, injury, or immunoinflammatory responses.

### Exemplary Methods

In certain embodiments, the invention relates to a method of increasing CXCL10 secretion by a cell, comprising the step of contacting the cell with any one of the inhibitors described herein.

In certain embodiments, the invention relates to any one of the aforementioned methods, wherein the inhibitor is an inhibitor of FAP, an inhibitor of DPPIV, an inhibitor of DPP8, or an inhibitor of DPP9. In certain embodiments, the invention relates to any one of the aforementioned methods, wherein the inhibitor is an inhibitor of FAP and an inhibitor of DPPIV. In certain embodiments, the invention relates to any one of the aforementioned methods, wherein the inhibitor is an inhibitor of FAP and an inhibitor of DPP8. In certain embodiments, the invention relates to any one of the aforementioned methods, wherein the inhibitor is an inhibitor of FAP and an inhibitor of DPP9. In certain embodiments, the invention relates to any one of the aforementioned methods, wherein the inhibitor is an inhibitor of DPP8 and an inhibitor of DPPIV. In certain embodiments, the invention relates to any one of the aforementioned methods, wherein the inhibitor is an inhibitor of DPP9 and an inhibitor of DPPIV. In certain embodiments, the invention relates to any one of the aforementioned methods, wherein the inhibitor is an inhibitor of DPP8 and an inhibitor of DPP9. In certain embodiments, the invention relates to any one of the aforementioned methods, wherein the inhibitor is an inhibitor of FAP, an inhibitor of DPPIV, and an inhibitor of DPP8. In certain embodiments, the invention relates to any one of the aforementioned methods, wherein the inhibitor is an inhibitor of FAP, an inhibitor of DPPIV, and an inhibitor of DPP9. In certain embodiments, the invention relates to any one of the aforementioned methods, wherein the inhibitor is an inhibitor of FAP, an inhibitor of DPP8, and an inhibitor of DPP9. In certain embodiments, the invention relates to any one of the aforementioned methods, wherein the inhibitor is an inhibitor of DPPIV, an inhibitor of DPP8, and an inhibitor of DPP9.

In certain embodiments, the invention relates to a method of increasing CXCL10 secretion by a cell, comprising the step of contacting the cell with an inhibitor of FAP, an inhibitor of DPPIV, an inhibitor of DPP8, or an inhibitor of DPP9.

In certain embodiments, the invention relates to a method of increasing CXCL10 secretion by a cell, comprising the step of contacting the cell with an inhibitor, wherein the inhibitor is an inhibitor of DPPIV and an inhibitor of DPP8 or DPP9.

In certain embodiments, the invention relates to a method of treating, inhibiting, or preventing a viral infection, comprising the step of administering to a mammal in need thereof a therapeutically or prophylactically effective amount of any one of the inhibitors described herein.

In certain embodiments, the invention relates to any one of the aforementioned methods, wherein the inhibitor is an inhibitor of FAP and an inhibitor of DPPIV. In certain embodiments, the invention relates to any one of the aforementioned methods, wherein the inhibitor is an inhibitor of FAP and an inhibitor of DPP8. In certain embodiments, the invention relates to any one of the aforementioned methods, wherein the inhibitor is an inhibitor of FAP and an inhibitor of DPP9. In certain embodiments, the invention relates to any one of the aforementioned methods, wherein the inhibitor is an inhibitor of DPP8 and an inhibitor of DPPIV. In certain embodiments, the invention relates to any one of the aforementioned methods, wherein the inhibitor is an inhibitor of DPP9 and an inhibitor of DPPIV. In certain embodiments, the invention relates to any one of the aforementioned methods, wherein the inhibitor is an inhibitor of DPP8 and an inhibitor of DPP9. In certain embodiments, the invention relates to any one of the aforementioned methods, wherein the inhibitor is an inhibitor of FAP, an inhibitor of DPPIV, and an inhibitor of DPP8. In certain embodiments, the invention relates to any one of the aforementioned methods, wherein the inhibitor is an inhibitor of FAP, an inhibitor of DPPIV, and an inhibitor of DPP9. In certain embodiments, the invention relates to any one of the aforementioned methods, wherein the inhibitor is an inhibitor of FAP, an inhibitor of DPP8, and an inhibitor of DPP9. In certain embodiments, the invention relates to any one of the aforementioned methods, wherein the inhibitor is an inhibitor of DPPIV, an inhibitor of DPP8, and an inhibitor of DPP9.

In certain embodiments, the invention relates to a method of treating, inhibiting, or preventing a viral infection, comprising the step of administering to a mammal in need thereof a therapeutically or prophylactically effective amount of an inhibitor of FAP, an inhibitor of DPPIV, an inhibitor of DPP8, or an inhibitor of DPP9.

In certain embodiments, the invention relates to a method of treating, inhibiting, or preventing a viral infection, comprising the step of administering to a mammal in need thereof a therapeutically or prophylactically effective amount of an inhibitor, wherein the inhibitor is an inhibitor of DPPIV and an inhibitor of DPP8 or DPP9.

In certain embodiments, the invention relates to any one of the aforementioned methods, wherein the viral infection is a viral infection of the liver.

In certain embodiments, the invention relates to any one of the aforementioned methods, wherein the viral infection is hepatitis B virus, hepatitis C virus, human immunodeficiency virus, Polio virus, Coxsackie A virus, Coxsackie B virus, Rhino virus, respiratory syncytial virus, dengue virus, equine infectious anemia virus, Echo virus, small pox virus, Ebola virus, or West Nile virus.

In certain embodiments, the invention relates to any one of the aforementioned methods, wherein the viral infection is hepatitis C virus.

In certain embodiments, the invention relates to any one of the aforementioned methods, wherein the mammal is a primate, equine, canine, feline, or bovine.

In certain embodiments, the invention relates to any one of the aforementioned methods, wherein the mammal is a human.

In certain embodiments, the invention relates to any one of the aforementioned methods, wherein the inhibitor is administered to the mammal by inhalation, orally, intravenously, sublingually, ocularly, transdermally, rectally, vaginally, topically, intramuscularly, intra-arterially, intrathecally, subcutaneously, buccally, or intranasally.

In certain embodiments, the invention relates to any one of the aforementioned methods, wherein the inhibitor is administered to the mammal intravenously.

In certain embodiments, the invention relates to any one of the aforementioned methods, wherein the inhibitor is administered to the mammal orally.

In certain embodiments, the invention relates to any one of the aforementioned methods, wherein the inhibitor is co-administered with a second agent. In certain embodiments, the second agent is a second antiviral agent. In certain embodiments, the second antiviral agent is selected from the group consisting of ribavirin, pegylated interferon alfa-2a, interferon alfacon-1, natural interferon, Albuferon (Human Genome Sciences), interferon beta-1a, omega interferon, oral interferon alpha, interferon gamma-1b, IP-501 (Interneuron), Merimebodib VX-497 (Vertex), Symmetrel (Endo), IDN-6556 (Idun), XTL-002 (XTL), HCV/MF59 (Chiron), Civacir (Nabi), Viramidine (ICN), thymosin alfa-1, histamine dihydrochloride, VX 950/LY 570310 (Vertex, Eli Lilly), ISIS 14803 (Isis), JTK 003 (Akros), Tarvacin, HCV-796 (Viro/Wye), CH-6 (Schering), ANA971 (Anadys), ANA245 (Anadys), Actilon, Rituxam, Valopicitabine, HepX-C(XTL), IC41 (Intercell), Medusa interferon (Flamel), E-1 (Innogenetics), Multiferon (Viragen), BILN 2061 (Boehringer Ingelheim), and REBIF (Ares-Serono).

### Exemplary Inhibitors

One aspect of the invention relates to methods using an inhibitor having a structure of Formula (I)

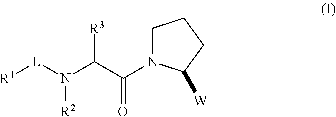

wherein

L is absent or is —XC(O)—;
R¹ is selected from H, C₁₋₆alkyl, C₁₋₆acyl, C₁₋₆aralkyl, C₁₋₆aracyl, C₁₋₆heteroaracyl, carbocyclyl, aryl, and ArSO₂—;
R² is selected from H and C₁₋₆alkyl, or R¹ and R² together are phthaloyl, thereby forming a ring;
R³ is selected from H, C₁₋₆alkyl, C₁₋₆hydroxyalkyl, C₁₋₆thioalkyl, and C₁₋₆aralkyl;
W is selected from B(Y¹)(Y²) and CN;
Y¹ and Y² are independently selected from OH or a group that is hydrolyzable to give a boronic acid, or together with the boron atom to which they are attached form a 5- to 8-membered ring that is hydrolyzable to a boronic acid;
X is selected from O and NH.

In certain embodiments, L is absent; and R¹ is selected from H, C₁₋₆alkyl, C₁₋₆acyl, C₁₋₆aralkyl, C₁₋₆aracyl, C₁₋₆heteroaracyl, carbocyclyl, aryl, and ArSO₂—. In certain such embodiments, L is absent; and R¹ is C₁₋₆alkyl selected from methyl, ethyl, isopropyl, and tert-butyl. In certain such embodiments, L is absent; and R¹ is C₁₋₆acyl selected from acetyl and pivaloyl. In certain such embodiments, L is absent; and R¹ is phenylmethyl. In certain such embodiments, L is absent; and R¹ is aracyl selected from 2-phenylethylcarbonyl, phenylmethylcarbonyl, (1-naphthyl)carbonyl, and (2-naphthyl)carbonyl, and (4-sulfamoylphenyl)carbonyl. In certain embodiments, L is absent; and R¹ is pyrazyl. In certain embodiments, L is absent; and R¹ carbocyclyl selected from cyclohexyl and adamantyl. In certain embodiments, L is absent; and R¹ is selected from phenyl and phenylsulfonyl.

In certain embodiments, L is —XC(O)—, X is O, and R¹ is C₁₋₆aralkyl. In certain such embodiments L is —XC(O)—, X is O, and R¹ is phenylmethyl.

In certain embodiments, L is —XC(O)—, X is NH, and R¹ is selected from aryl and C₁₋₆aralkyl. In certain embodiments, L is —XC(O)—, X is NH, and R¹ is selected from phenyl and phenylmethyl.

In certain embodiments, R² is C₁₋₆alkyl. In certain embodiments, R¹ is selected from methyl, isopropyl, and t-butyl. In certain embodiments, R¹ is methyl.

Another aspect of the invention relates to methods using an inhibitor having a structure of Formula II

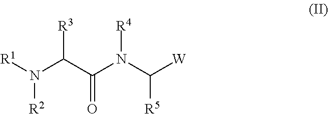

wherein
R¹ is selected from H, C₁₋₆alkyl, C₁₋₆acyl, C₁₋₆aralkyl, C₁₋₆aracyl, C₁₋₆heteroaracyl, and carbocyclyl;
R² is selected from H and C₁₋₆alkyl;
R³ is selected from H, C₁₋₆alkyl, C₁₋₆hydroxyalkyl, C₁₋₆thioalkyl, and C₁₋₆aralkyl;
R⁴ is selected from H and C₁₋₆alkyl, or R³ and R⁴ together are C₁₋₆alkyl thereby forming a ring;
R⁵ is selected from H, C₁₋₆alkyl, C₁₋₆hydroxyalkyl, C₁₋₆thioalkyl, and C₁₋₆aralkyl, or R⁴ and R⁵ together are C₁₋₆alkyl-S—C₁₋₆alkyl;
W is selected from H, B(Y¹)(Y²), and CN;
Y¹ and Y² are independently selected from OH or a group that is hydrolyzable to give a boronic acid, or together with the boron atom to which they are attached form a 5- to 8-membered ring that is hydrolysable to a boronic acid;
   with the proviso that W can be H only when R⁴ and R⁵ together are C₁₋₆alkyl-S—C₁₋₆alkyl.

In certain embodiments, R¹ is selected from C₁₋₆acyl and C₁₋₆aracyl. In certain embodiments, R¹ is selected from phenylcarbonyl, (1-naphthyl)carbonyl, and acetyl.

In certain embodiments, R⁵ is C₁₋₆alkyl. In certain embodiments, R⁵ is selected from methyl and ethyl.

In certain embodiments, W is H; and R⁴ and R⁵ together are C₁₋₆alkyl-S—C₁₋₆alkyl. In certain embodiments, W is H; and R⁴ and R⁵ together are C₂alkyl-S—C₁alkyl, thereby forming a five-membered ring.

In certain embodiments, R³ and R⁴ together are C₁₋₆alkyl thereby forming a ring. In certain embodiments, R³ and R⁴ together are C₂alkyl, thereby forming a five-membered ring.

Another aspect of the invention relates to methods using an inhibitor selected from the group consisting of:

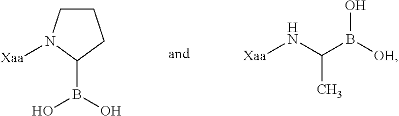

wherein Xaa is a natural or non-natural amino acid.

Another aspect of the invention relates to methods using an inhibitor selected from the group consisting of:

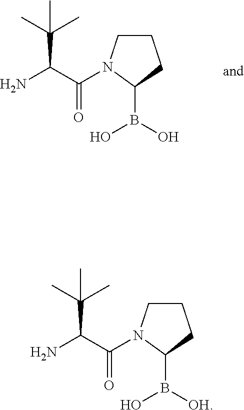 [SMILES: CC(C)(C)[C@H](N)C(=O)N1CCC[C@H]1B(O)O ; CC(C)(C)[C@H](N)C(=O)N1CCC[C@H]1B(O)O]

In certain embodiments, the invention relates to any one of the aforementioned methods, wherein the inhibitor is described in U.S. Patent Application Publication No. 2010/0184706, U.S. Patent Application Publication No. 2010/0105753, U.S. Patent Application Publication No. 2010/0105629, U.S. Patent Application Publication No. 2009/0209491, U.S. Patent Application Publication No. 2009/0124559, U.S. Patent Application Publication No. 2005/0203027, U.S. Pat. No. 7,998,997, U.S. Pat. No. 7,727,964, U.S. Pat. No. 7,691,967, U.S. Pat. No. 7,459,428, U.S. Pat. No. 6,011,155, U.S. Pat. No. 7,399,869, U.S. Pat. No. 8,183,280, U.S. Patent Application Publication No. 2008/0057491, U.S. Patent Application Publication No. 2008/0280856, U.S. Patent Application Publication No. 2010/0047170, U.S. Patent Application Publication No. 2011/0112069, U.S. Patent Application Publication No. 2011/0144037, U.S. Patent Application Publication No. 2008/0175837, or U.S. Patent Application Publication No. 2009/0137457, the contents of each of which are incorporated by reference in their entirety.

In certain embodiments, the invention relates to any one of the aforementioned methods, wherein the inhibitor is sitagliptin, vildagliptin, saxagliptin, linagliptin, dutoglipin, gemigliptin, alogliptin, denagliptin, ABT-341, or berberine.

In certain embodiments, the invention relates to any one of the aforementioned methods, wherein the inhibitor is talabostat or sibrotuzumab.

In certain embodiments, the invention relates to any one of the aforementioned methods, wherein the inhibitor has an IC₅₀ less than about 500 nM, less than about 400 nM, less than about 300 nM, less than about 200 nM, less than about 100 nM, less than about 75 nM, less than about 50 nM, less than about 40 nM, less than about 30 nM, less than about 20 nM, less than about 10 nM, or less than about 5 nM. In certain embodiments, the invention relates to any one of the aforementioned methods, wherein the inhibitor has an IC₅₀ greater than about 0.01 nM.

### Exemplary Compositions

Another aspect of the present invention relates to a pharmaceutical composition, comprising an inhibitor described herein; and a pharmaceutically acceptable excipient.

Another aspect of the present invention relates to a pharmaceutical composition, comprising a pharmaceutically acceptable carrier; and a prodrug of any one of the inhibitors disclosed herein.

Hosts, including but not limited to humans, infected with hepatitis C virus (“HCV”), or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound will be in the range of between about 0.1 and about 100 mg/kg, more generally, between about 1 and 50 mg/kg, and, preferably, between about 1 and about 20 mg/kg, of body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3,000 mg, preferably 70 to 1400 mg of active ingredient per unit dosage form. An oral dosage of 50-1,000 mg is usually convenient.

Ideally the active ingredient should be administered to achieve peak plasma concentrations of the active compound from about 0.2 to 70 μM, preferably about 1.0 to 15 μM. This can be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### Exemplary Uses

Another aspect of the present invention relates to the use of any one of the inhibitors disclosed herein in the manufacture of a medicament for the treatment of a viral infection.

Another aspect of the present invention relates to the use of a prodrug of any one of the inhibitors disclosed herein in the manufacture of a medicament for the treatment of a viral infection.

### Exemplary Packaged Pharmaceuticals

Another aspect of the present invention relates to a packaged pharmaceutical, comprising any one of the inhibitors disclosed herein formulated in a pharmaceutically acceptable excipient, in association with instructions (written and/or pictorial) describing the recommended dosage and/or administration of the formulation to a patient.

Another aspect of the present invention relates to a packaged pharmaceutical, comprising a prodrug of any of the inhibitors disclosed herein formulated in a pharmaceutically acceptable excipient, in association with instructions (written and/or pictorial) describing the recommended dosage and/or administration of the formulation to a patient.

### DEFINITIONS

The term “amino acid” is intended to embrace all compounds, whether natural or synthetic, which include both an amino functionality and an acid functionality, including amino acid analogues and derivatives. In certain embodiments, the amino acids contemplated in the present invention are those naturally occurring amino acids found in proteins, or the naturally occurring anabolic or catabolic products of such amino acids, which contain amino and carboxyl groups. Naturally occurring amino acids are identified throughout by the conventional three-letter and/or one-letter abbreviations, corresponding to the trivial name of the amino acid, in accordance with the following list. All amino acids described herein are contemplated as both (D)- and (L)-isomers unless otherwise designated. The abbreviations are accepted in the peptide art and are recommended by the IUPAC-IUB commission in biochemical nomenclature.

By the term “amino acid residue” is meant an amino acid. In general the abbreviations used herein for designating the naturally occurring amino acids are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group.

The term “amino acid side chain” is that part of an amino acid residue exclusive of the backbone, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H— (the side chain of glycine). These sidechains are pendant from the backbone Cα carbon.

The term “amino acid analog” refers to a compound structurally similar to a naturally occurring amino acid wherein the C-terminal carboxy group, the N-terminal amino group or side-chain functional group has been chemically modified. For example, aspartic acid-(beta-methyl ester) is an amino acid analog of aspartic acid; N-ethylglycine is an amino acid analog of glycine; or alanine carboxamide is an amino acid analog of alanine.

The phrase “protecting group” as used herein means substituents which protect the reactive functional group from undesirable chemical reactions. Examples of such protecting groups include esters of carboxylic acids and boronic acids, ethers of alcohols, and acetals and ketals of aldehydes and ketones. For instance, the phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as, to illustrate, formyl, dansyl, acetyl, benzoyl, trifluoroacetyl, succinyl, and methoxysuccinyl; aromatic urethane protecting groups as, for example, benzyloxycarbonyl (Cbz); and aliphatic urethane protecting groups such as t-butoxycarbonyl (Boc) or 9-Fluorenylmethoxycarbonyl (Fmoc).

The term “amino-terminal protecting group” as used herein, refers to terminal amino protecting groups that are typically employed in organic synthesis, especially peptide synthesis. Any of the known categories of protecting groups can be employed, including acyl protecting groups, such as acetyl, and benzoyl; aromatic urethane protecting groups, such as benzyloxycarbonyl; and aliphatic urethane protecting groups, such as tert-butoxycarbonyl. See, for example, Gross and Mienhoffer, Eds., The Peptides, Academic Press: New York, 1981; Vol. 3, 3-88; and Green, T. W.; Wuts, P. G. M., Protective Groups in Organic Synthesis, 2nd ed, Wiley: New York, 1991. Preferred protecting groups include aryl-, aralkyl-, heteroaryl- and heteroarylalkyl-carbonyl and sulfonyl moieties.

As used herein the term “physiological conditions” refers to temperature, pH, ionic strength, viscosity, and like biochemical parameters which are compatible with a viable organism, and/or which typically exist intracellularly in a viable mammalian cell

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As noted above, certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer.

“Cycloalkyl” means mono- or bicyclic or bridged saturated carbocyclic rings, each having from 3 to 12 carbon atoms. Likewise, preferred cycloalkyls have from 5-12 carbon atoms in their ring structure, and more preferably have 6-10 carbons in the ring structure.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywherein the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkynyl, and —(S)—(CH₂)ₘ—R¹, wherein m and R¹ are defined below. Representative alkylthio groups include methylthio, ethylthio, and the like.

The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O— alkenyl, —O-alkynyl, —O—(CH₂)ₘ—R¹, where m and R₁ are described below.

The terms “amine” and “amino” are art-recognized and refer to both unsubstituted and substituted amines, e.g., a moiety that can be represented by the formulae:

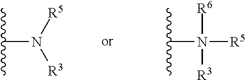

wherein R³, R⁵ and R⁶ each independently represent a hydrogen, an alkyl, an alkenyl, —(CH₂)ₘ—R¹, or R³ and R⁵ taken together with the N atom to which they are attached complete a heterocycle having from 4 to 8 atoms in the ring structure; R¹ represents an alkenyl, aryl, cycloalkyl, a cycloalkenyl, a heterocyclyl, or a polycyclyl; and m is zero or an integer in the range of 1 to 8. In certain embodiments, only one of R³ or R⁵ can be a carbonyl, e.g., R³, R⁵, and the nitrogen together do not form an imide. In even more certain embodiments, R³ and R⁵ (and optionally R⁶) each independently represent a hydrogen, an alkyl, an alkenyl, or —(CH₂)ₘ—R¹. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 6- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like.

The terms “heterocyclyl” or “heterocyclic group” refer to 3- to 12-membered ring structures, more preferably 5- to 12-membered rings, more preferably 6- to 10-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “carbonyl” is art-recognized and includes such moieties as can be represented by the formula:

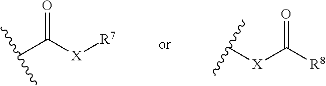

wherein X is a bond or represents an oxygen or a sulfur, and R⁷ represents a hydrogen, an alkyl, an alkenyl, —(CH₂)ₘ—R¹ or a pharmaceutically acceptable salt, R⁸ represents a hydrogen, an alkyl, an alkenyl or —(CH₂)ₘ—R¹, where m and R¹ are as defined above. Where X is an oxygen and R⁷ or R⁸ is not hydrogen, the formula represents an “ester.” Where X is an oxygen, and R⁷ is as defined above, the moiety is referred to herein as a carboxyl group, and particularly when R⁷ is a hydrogen, the formula represents a “carboxylic acid”. Where X is an oxygen, and R⁸ is a hydrogen, the formula represents a “formate.” In general, where the oxygen atom of the above formula is replaced by a sulfur, the formula represents a “thiocarbonyl” group. Where X is a sulfur and R⁷ or R⁸ is not hydrogen, the formula represents a “thioester” group. Where X is a sulfur and R⁷ is a hydrogen, the formula represents a “thiocarboxylic acid” group. Where X is a sulfur and R⁸ is a hydrogen, the formula represents a “thioformate” group. On the other hand, where X is a bond, and R⁷ is not hydrogen, the above formula represents a “ketone” group. Where X is a bond, and R⁷ is a hydrogen, the above formula represents an “aldehyde” group.

The term “thioxamide,” as used herein, refers to a moiety that can be represented by the formula:

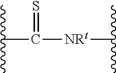

in which R^{t} is selected from the group consisting of the group consisting of hydrogen, alkyl, cycloalkyl, aralkyl, or aryl, preferably hydrogen or alkyl. Moreover, “thioxamide-derived” compounds or “thioxamide analogs” refer to compounds in which one or more amide groups have been replaced by one or more corresponding thioxamide groups. Thioxamides are also referred to in the art as “thioamides.”

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The term “sulfamoyl” is art-recognized and includes a moiety that can be represented by the formula:

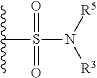

in which R³ and R⁵ are as defined above.

The term “sulfate” is art recognized and includes a moiety that can be represented by the formula:

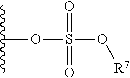

in which R⁷ is as defined above.

The term “sulfonamide” is art recognized and includes a moiety that can be represented by the formula:

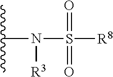

in which R³ and R⁸ are as defined above.

The term “sulfonate” is art-recognized and includes a moiety that can be represented by the formula:

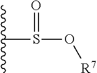

in which R⁷ is an electron pair, hydrogen, alkyl, cycloalkyl, or aryl.

The terms “sulfoxido” or “sulfinyl”, as used herein, refers to a moiety that can be represented by the formula:

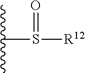

in which R¹² is selected from the group consisting of the group consisting of hydrogen, alkyl, alkenyl, alkynyl, cycloalkyl, heterocyclyl, aralkyl, or aryl.

As used herein, the definition of each expression, e.g., alkyl, m, n, etc., when it occurs more than once in any structure, is intended to be independent of its definition elsewherein the same structure.

For purposes of this invention, the chemical elements are identified in accordance with the Periodic Table of the Elements, CAS version, Handbook of Chemistry and Physics, 67th ed., 1986-87, inside cover.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

All of the U.S. patents and U.S. patent application publications cited herein are hereby incorporated by reference.

1. (canceled)

2. A method of treating, inhibiting, or preventing a viral infection, comprising the step of administering to a mammal in need thereof a therapeutically or prophylactically effective amount of an inhibitor of fibroblast activation protein (FAP), an inhibitor of dipeptidyl peptidase type 4 (DPPIV), an inhibitor of dipeptidyl peptidase type 8 (DPP8) or an inhibitor of dipeptidyl peptidase type 9 (DPP9).

3. The method of claim 2, wherein the inhibitor is an inhibitor of FAP and an inhibitor of DPPIV.

4. The method of claim 2, wherein the inhibitor is an inhibitor of FAP and an inhibitor of DPP8.

5. The method of claim 2, wherein the inhibitor is an inhibitor of FAP and an inhibitor of DPP9.

6. The method of claim 2, wherein the inhibitor is an inhibitor of DPP8 and an inhibitor of DPPIV.

7. The method of claim 2, wherein the inhibitor is an inhibitor of DPP9 and an inhibitor of DPPIV.

8. The method of claim 2, wherein the inhibitor is an inhibitor of DPP8 and an inhibitor of DPP9.

9. The method of claim 2, wherein the inhibitor is an inhibitor of FAP, an inhibitor of DPPIV, and an inhibitor of DPP8.

10. The method of claim 2, wherein the inhibitor is an inhibitor of FAP, an inhibitor of DPPIV, and an inhibitor of DPP9.

11. The method of claim 2, wherein the inhibitor is an inhibitor of FAP, an inhibitor of DPP8, and an inhibitor of DPP9.

12. The method of claim 2, wherein the inhibitor is an inhibitor of DPPIV, an inhibitor of DPP8, and an inhibitor of DPP9.

13. The method of claim 2, wherein the viral infection is a viral infection of the liver.

14. The method of claim 2, wherein the viral infection is hepatitis B virus, hepatitis C virus, human immunodeficiency virus, Polio virus, Coxsackie A virus, Coxsackie B virus, Rhino virus, respiratory syncytial virus, dengue virus, equine infectious anemia virus, Echo virus, small pox virus, Ebola virus, or West Nile virus.

15. The method of claim 2, wherein the viral infection is hepatitis C virus.

16. (canceled)

17. The method of claim 2, wherein the mammal is a human.

18. The method of claim 2, wherein the inhibitor is administered to the mammal by inhalation, orally, intravenously, sublingually, ocularly, transdermally, rectally, vaginally, topically, intramuscularly, intra-arterially, intrathecally, subcutaneously, buccally, or intranasally.

19. The method of claim 2, wherein the inhibitor is administered to the mammal intravenously.

20. The method of claim 2, wherein the inhibitor is administered to the mammal orally.

21. The method of claim 2, wherein the inhibitor is co-administered with a second agent.

22. The method of claim 21, wherein the second agent is a second antiviral agent.

23. The method of claim 22, wherein the second antiviral agent is selected from the group consisting of ribavirin, pegylated interferon alfa-2a, interferon alfacon-1, natural interferon, Albuferon, interferon beta-1a, omega interferon, oral interferon alpha, interferon gamma-1b, IP-501, Merimebodib VX-497, Symmetrel, IDN-6556, XTL-002, HCV/MF59, Civacir, Viramidine, thymosin alfa-1, histamine dihydrochloride, VX 950/LY 570310, ISIS 14803, JTK 003, Tarvacin, HCV-796, CH-6, ANA971, ANA245, Actilon, Rituxan, Valopicitabine, HepX-C, IC41, Medusa interferon, E-1, Multiferon, BILN 2061, and REBIF.

24. The method of claim 2, wherein the inhibitor has a structure of Formula (I) 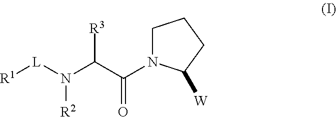
wherein
L is absent or is —XC(O)—;
R¹ is selected from H, C₁₋₆alkyl, C₁₋₆acyl, C₁₋₆aralkyl, C₁₋₆aracyl, C₁₋₆heteroaracyl, carbocyclyl, aryl, and ArSO₂—;
R² is selected from H and C₁₋₆alkyl, or R¹ and R² together are phthaloyl, thereby forming a ring;
R³ is selected from H, C₁₋₆alkyl, C₁₋₆hydroxyalkyl, C₁₋₆thioalkyl, and C₁₋₆aralkyl;
W is selected from B(Y¹)(Y²) and CN;
Y¹ and Y² are independently selected from OH or a group that is hydrolyzable to give a boronic acid, or together with the boron atom to which they are attached form a 5- to 8-membered ring that is hydrolyzable to a boronic acid; and
X is selected from O and NH.

25. The method of claim 2, wherein the inhibitor has a structure of Formula (II) 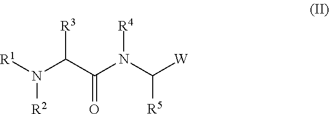
wherein
R¹ is selected from H, C₁₋₆alkyl, C₁₋₆acyl, C₁₋₆aralkyl, C₁₋₆aracyl, C₁₋₆heteroaracyl, and carbocyclyl;
R² is selected from H and C₁₋₆alkyl;
R³ is selected from H, C₁₋₆alkyl, C₁₋₆hydroxyalkyl, C₁₋₆thioalkyl, and C₁₋₆aralkyl;
R⁴ is selected from H and C₁₋₆alkyl, or R³ and R⁴ together are C₁₋₆alkyl thereby forming a ring;
R⁵ is selected from H, C₁₋₆alkyl, C₁₋₆hydroxyalkyl, C₁₋₆thioalkyl, and C₁₋₆aralkyl, or R⁴ and R⁵ together are C₁₋₆alkyl-S—C₁₋₆alkyl;
W is selected from H, B(Y¹)(Y²), and CN; and
Y¹ and Y² are independently selected from OH or a group that is hydrolyzable to give a boronic acid, or together with the boron atom to which they are attached form a 5- to 8-membered ring that is hydrolyzable to a boronic acid;
with the proviso that W can be H only when R⁴ and R⁵ together are C₁₋₆alkyl-S—C₁₋₆alkyl.

26. The method of claim 2, wherein the inhibitor is selected from the group consisting of: 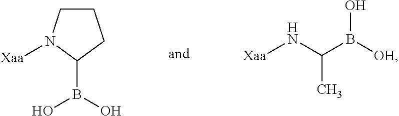
wherein Xaa is a natural or non-natural amino acid.

27. The method of claim 2, wherein the inhibitor is selected from the group consisting of:

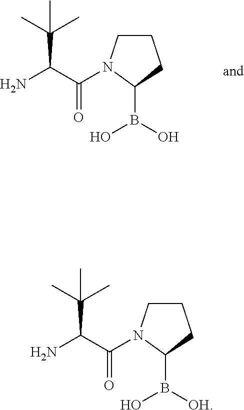 [SMILES: CC(C)(C)[C@H](N)C(=O)N1CCC[C@H]1B(O)O ; CC(C)(C)[C@H](N)C(=O)N1CCC[C@H]1B(O)O]

28. The method of claim 27, wherein the inhibitor is

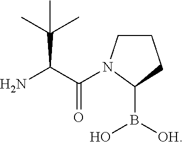 [SMILES: CC(C)(C)[C@H](N)C(=O)N1CCC[C@H]1B(O)O]

29. The method of claim 27, wherein the inhibitor is co-administered with a second agent; and said second agent is selected from the group consisting of ribavirin and pegylated interferon alfa-2a.

30. The method of claim 2, wherein the inhibitor is selected from the group consisting of sitagliptin, vildagliptin, saxagliptin, linagliptin, dutogliptin, gemigliptin, alogliptin, and berberine.

31. (canceled)

32. (canceled)

33. (canceled)
